(19) United States
(12) Patent Application Publication
(10) Pub. No.: US 2006/0217371 A1
(43) Pub. Date: Sep. 28, 2006

(54) Diazabicyclononene and tetrahydropyriddine derivatives as renin inhibitors

(76) Inventors: Olivier Bezencon, Riehen (CH); Daniel Bur, Therwil (CH); Walter Fischli, Obertorweg (CH); Lucos Remen, Allschwill (CH); Sylvia Richard-Bildstein, Dietwiller (FR); Thomas Weller, Binningen (CH)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/554,443

(22) PCT Filed: Apr. 26, 2004
(86) PCT No.: PCT/EP04/04373

§ 371 Date: Oct. 25, 2005

(30) Foreign Application Priority Data
Apr. 28, 2003 (WO) PCT/EP03/04393

(51) Int. Cl. A61K 31/551; A61K 31/498; C07D 487/04

(52) U.S. Cl. 514/221; 514/249; 540/567; 544/349

(57) ABSTRACT

The invention relates to novel 3,9-diazabicyclo[3.3.1]nonene derivatives, tetrahydropyridine derivatives, and related compounds and their use as active ingredients in the preparation of pharmaceutical compositions. The invention also concerns related aspects including processes for the preparation of the compounds, pharmaceutical compositions containing one or more of those compounds and especially their use as inhibitors of renin.

The invention relates to novel compounds of the general formula I. The invention also concerns related aspects including processes for the preparation of the compounds, pharmaceutical compositions containing one or more compounds of formula I and especially their use as renin inhibitors in cardiovascular events and renal insufficiency. Furthermore, these compounds can be regarded as inhibitors of other aspartyl proteases and might therefore be useful as inhibitors of plasmepsins to treat malaria and as inhibitors of Candida albicans secreted aspartyl proteases to treat fungal infections.

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med, 1988, 84 (Suppl. 3A), 83) and myocardial infarction (Pfeffer M. A. et al., N. Engl. J. Med, 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes to Ang II, whose concentration is dramatically increased by the blockade of AT₁ receptors. This may raise serious questions regarding the safety and efficacy profile of AT₁ receptor antagonists. In summary, renin inhibitors are not only expected to be different from ACE inhibitors and AT₁ blockers with regard to safety, but more importantly also with regard to their efficacy to block the RAS.

Only limited clinical experience (Azizi M. et al, J. Hypertens., 1994, 12, 419; Neutel J. M. et al., Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only one compound containing four chiral centers has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are missing and sought. Recently, the first non-peptide renin inhibitors were described which show high in vitro activity (Oefner C. et al, Chem. Biol., 1999, 6, 127; Patent Application WO97/09311; Märki H. P. et al., Il Farmaco, 2001, 56, 21). However, the development status of these compounds is not known.

The present invention relates to the identification of renin inhibitors of a non-peptidic nature and of low molecular weight. Orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis are described.

The present invention describes non-peptidic renin inhibitors.

In particular, the present invention relates to novel compounds of the general formula I, 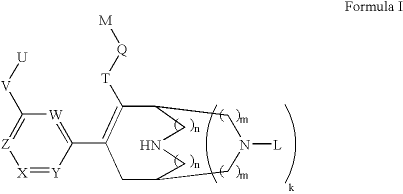
wherein
Z, Y, X and W represent independently a nitrogen atom or a —CH— group; at least two of the Z, Y, X and W represent a —CH— group;
V represents a bond; —(CH₂)ᵣ; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; or —CH₂—CH₂-A-CH₂—CH₂—B—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; or —COO—;
Q represents lower alkylene; lower alkenylene;
M represents hydrogen; cycloalkyl; aryl; heterocyclyl; heteroaryl;
L represents —R³; —COR³; —COOR³; —CONR²R³; —SO₂R³; —SO₂NR²R³; —COCH(Aryl)₂;
R¹ represents hydrogen; lower alkyl; lower alkenyl; lower alkinyl; cycloalkyl; aryl; cycloalkyl-lower alkyl;
R² and R²′ independently represent hydrogen; lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted or mono-, di- or trisubstituted with hydroxy, —OCOR², —COOR², lower alkoxy, cyano, —CONR²R²′, —CO-morpholin-4-yl, —CO-((4-loweralkyl)piperazin-1-yl), —NH(NH)NH₂, —NR⁴R⁴′ or lower alkyl, with the proviso that a carbon atom is attached at the most to one heteroatom in case this carbon atom is sp3-hybridized;
R⁴ and R⁴′ independently represent hydrogen; lower alkyl; cycloalkyl; cycloalkyl-lower alkyl; hydroxy-lower alkyl; —COOR²; —CONH₂;
k is the integer 0 or 1;
m and n represent the integer 0 or 1, with the proviso that in case m represents the integer 1, n is the integer 0; in case n represents the integer 1, m is the integer 0; in case k represents the integer 0, n represents the integer 0;
p is the integer 1, 2, 3 or 4;
r is the integer 1, 2, 3, 4, 5, or 6;
s is the integer 1, 2, 3, 4, or 5;
t is the integer 1, 2, 3, or 4;
u is the integer 1, 2, or 3;
v is the integer 2, 3, or 4;
and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

In the definitions of general formula I—if not otherwise stated—the term lower alkyl, alone or in combination with other groups, means saturated, straight and branched chain groups with one to seven carbon atoms, preferably one to four carbon atoms that can be optionally substituted by halogens. Examples of lower alkyl groups are methyl, ethyl, n-propyl, iso-propyl, n-butyl, iso-butyl, sec-butyl, tert-butyl, pentyl, hexyl and heptyl. The methyl, ethyl and isopropyl groups are preferred.

The term lower alkoxy refers to a R—O-group, wherein R is a lower alkyl. Examples of lower alkoxy groups are methoxy, ethoxy, propoxy, iso-propoxy, iso-butoxy, sec-butoxy and tert-butoxy.

The term lower alkenyl, alone or in combination with other groups, means straight and branched chain groups comprising an olefinic bond and consisting of two to seven carbon atoms, preferably two to four carbon atoms, that can be optionally substituted by halogens. Examples of lower alkenyl are vinyl, propenyl or butenyl.

The term lower alkinyl, alone or in combination with other groups, means straight and branched chain groups comprising a triple bond and consisting of two to seven carbon atoms, preferably two to four carbon atoms, that can be optionally substituted by halogens. Examples of lower alkinyl are ethinyl, propinyl or butinyl.

The term lower alkylene, alone or in combination with other groups, means straight and branched divalent chain groups with one to seven carbon atoms, preferably one to four carbon atoms, that can be optionally substituted by halogens. Examples of lower alkylene are ethylene, propylene or butylene.

The term lower alkenylene, alone or in combination with other groups, means straight and branched divalent chain groups comprising an olefinic bond and consisting of two to seven carbon atoms, preferably two to four carbon atoms, that can be optionally substituted by halogens. Examples of lower alkenylene are vinylene, propenylene and butenylene.

The term lower alkylenedioxy, refers to a lower alkylene substituted at each end by an oxygen atom. Examples of lower alkylenedioxy groups are preferably methylenedioxy and ethylenedioxy.

The term lower alkylenoxy refers to a lower alkylene substituted at one end by an oxygen atom. Examples of lower alkylenoxy groups are preferably methylenoxy, ethylenoxy and propylenoxy.

The term halogen means fluorine, chlorine, bromine or iodine, preferably fluorine, chlorine and bromine.

The term cycloalkyl alone or in combination, means a saturated cyclic hydrocarbon ring system with 3 to 7 carbon atoms, e.g. cyclopropyl, cyclobutyl, cyclopentyl, cyclohexyl and cycloheptyl, which can be optionally mono- or multisubstituted by lower alkyl, lower alkenyl, lower alkenylene, lower alkoxy, lower alkylenoxy, lower alkylenedioxy, hydroxy, halogen, —CF₃, —NR¹R¹′, —NR¹C(O)R¹′, —NR¹S(O₂)R¹′, —C(O)NR¹R¹′, lower alkylcarbonyl, —COOR¹, —SR¹, —SOR¹, —SO₂R¹, —SO₂NR¹R¹′ whereby R¹′ represents hydrogen; lower alkyl; lower alkenyl; lower alkinyl; cycloalkyl; aryl; cycloalkyl-lower alkyl. The cyclopropyl group is a preferred group.

The term aryl, alone or in combination, relates to the phenyl, the naphthyl or the indanyl group, preferably the phenyl group, which can be optionally mono- or multisubstituted by lower alkyl, lower alkenyl, lower alkinyl, lower alkenylene or lower alkylene forming with the aryl ring a five- or six-membered ring, lower alkoxy, lower alkylenedioxy, lower alkylenoxy, hydroxy, hydroxy-lower alkyl, halogen, cyano, —CF₃, —OCF₃, —NR¹R¹′, —NR¹R¹′-lower alkyl, —NR¹C(O)R¹′, —NR₁S(O₂)R¹, —C(O)NR¹R¹′, —NO₂, lower alkylcarbonyl, —COOR¹, —SR¹, —SOR¹, —SO₂R¹, —SO₂NR¹R¹′, benzyloxy, whereby R¹′ has the meaning given above.

The term aryloxy refers to an Ar—O-group, wherein Ar is an aryl. An example of a lower aryloxy group is phenoxy.

The term heterocyclyl, alone or in combination, means saturated or unsaturated (but not aromatic) five-, six- or seven-membered rings containing one or two nitrogen, oxygen or sulfur atoms which may be the same or different and which rings can be optionally substituted with lower alkyl, hydroxy, lower alkoxy and halogen. The nitrogen atoms, if present, can be substituted by a —COOR² group. Examples of such rings are piperidinyl, morpholinyl, thiomorpholinyl, piperazinyl, tetrahydropyranyl, dihydropyranyl, 1,4-dioxanyl, pyrrolidinyl, tetrahydrofiranyl, dihydropyrrolyl, imidazolidinyl, dihydropyrazolyl, pyrazolidinyl, dihydroquinolinyl, tetrahydroquinolinyl, tetrahydroisoquinolinyl.

The term heteroaryl, alone or in combination, means six-membered aromatic rings containing one to four nitrogen atoms; benzofused six-membered aromatic rings containing one to three nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing one oxygen and one nitrogen atom and to benzofused derivatives thereof; five-membered aromatic rings containing a sulfur and a nitrogen or an oxygen atom and benzofused derivatives thereof; five-membered aromatic rings containing two nitrogen atoms and benzofused derivatives thereof; five-membered aromatic rings containing three nitrogen atoms and benzofused derivatives thereof, or a tetrazolyl ring. Examples of such ring systems are furanyl, thiophenyl, pyrrolyl, pyridinyl, pyrimidinyl, indolyl, quinolinyl, isoquinolinyl, imidazolyl, triazinyl, thiazinyl, thiazolyl, isothiazolyl, pyridazinyl, pyrazolyl, oxazolyl, isoxazolyl, coumarinyl, benzothiophenyl, quinazolinyl, quinoxalinyl. Such rings may be adequatly substituted with lower alkyl, lower alkenyl, lower alkinyl, lower alkylene, lower alkenylene, lower alkylenedioxy, lower alkyleneoxy, hydroxy-lower alkyl, lower alkoxy, hydroxy, halogen, cyano, —CF₃, —OCF₃, —NR¹R¹′, —NR¹R¹′-lower alkyl, —N(R¹)COR¹, —N(R¹)SO₂R¹, —CONR¹R¹′, —NO₂, lower alkylcarbonyl, —COOR¹, —SR¹, —SOR¹, —SO₂R¹, —SO₂NR¹R¹′, another aryl, another heteroaryl or another heterocyclyl and the like, whereby R¹′ has the meaning given above.

The term heteroaryloxy refers to a Het-O-group, wherein Het is a heteroaryl.

The term sp3-hybridized refers to a carbom atom and means that this carbon atom forms four bonds to four substituents placed in a tetragonal fashion around this carbon atom.

The expression pharmaceutically acceptable salts encompasses either salts with inorganic acids or organic acids like hydrochloric or hydrobromic acid, sulfuric acid, phosphoric acid, citric acid, formic acid, acetic acid, maleic acid, tartaric acid, benzoic acid, methanesulfonic acid, p-toluenesulfonic acid, and the like that are non toxic to living organisms or in case the compound of formula I is acidic in nature with an inorganic base like an alkali or earth alkali base, e.g. sodium hydroxide, potassium hydroxide, calcium hydroxide and the like.

Compounds of the invention also include nitrosated compounds of the general to formula I that have been nitrosated through one or more sites such as oxygen (hydroxyl condensation), sulfur (sulffiydryl condensation) and/or nitrogen. The nitrosated compounds of the present invention can be prepared using conventional methods known to one skilled in the art. For example, known methods for nitrosating compounds are described in U.S. Pat. Nos. 5,380,758 and 5,703,073; WO 97/27749; WO 98/19672; WO 98/21193; WO 99/00361 and Oae et al, Org. Prep. Proc. Int., 15(3):165-198 (1983), the disclosures of each of which are incorporated by reference herein in their entirety.

The compounds of the general formula I can contain two or more asymmetric carbon atoms and may be prepared in form of optically pure enantiomers, mixtures of enantiomers such as racemates, diastereomers, mixtures of diastereomers, diastereomeric racemates, mixtures of diastereomeric racemates, and the meso-form and pharmaceutically acceptable salts thereof.

The present invention encompasses all these forms. Mixtures may be separated in a manner known per se, i.e. by column chromatography, thin layer chromatography, HPLC or crystallization.

A group of preferred compounds are compounds of general formula I wherein Z, Y, X, W, V, U, T, Q, L, and M are as defined in general formula I above and wherein

k is 1

n is 0 and

m is 1.

Another group of preferred compounds of general formula I are those wherein Z, Y, X, W, V, U, T, Q, M, k, m, and n are as defined in general formula I above and

L represents H; —COR³″; —COOR³″; —CONR²″R³″;

whereby R²″ and R³″ represent independently lower alkyl, lower cycloalkyl-lower alkyl, which lower alkyl and lower cycloalkyl-lower alkyl groups are unsubstituted or monosubstituted with halogen, cyano, hydroxy, —OCOCH₃, —CONH₂, —COOH or —NH₂, with the proviso that a carbon atom is attached at the most to one heteroatom in case this carbon atom is sp3-hybridized.

Another group of preferred compounds of general formula I above are those wherein Z, Y, X, W, V, U, L, k, m, and n are as defined in general formula I and

T is —CONR¹—;

Q is methylene;

M is aryl; heteroaryl.

Another group of also more preferred compounds of general formula I are those wherein V, U, T, Q, M, L, k, m, and n are as defined in general formula I above and

Z, Y, X and W represent —CH—.

Another group of also more preferred compounds of general formula I are those wherein Z, Y, X, W, V, Q, T, M, L, k, m, and n are as defined in general formula I above and

U is a mono-, di-, or trisubstituted phenyl or heteroaryl, wherein the substituents are halogen, lower alkyl, lower alkoxy, CF₃.

Especially preferred compounds of general formula I are those selected from the group consisting of:
(rac.)-(1R*, 5S*)-(3-acetyl-7-{3-[2-(2-bromo-5-fluorophenoxy)ethyl]phenyl}-3,9-diazabicyclo[3.3.1]non-6-ene-6-carboxylic acid methylphenethylamide;
(rac.)-(1R*,5S*)-3-acetyl-7-{3-[2-(2-chlorophenoxy)ethyl]phenyl}-3,9-diazabicyclo[3.3.1]non-6-ene-6-carboxylic acid methylphenethylamide;
(rac.)-(1R*, 5S*)-3-acetyl-7-{3-[2-(2-tert-butylphenoxy)ethyl]phenyl}-3,9-diazabicyclo[3.3.1]non-6-ene-6-carboxylic acid methylphenethylamide;
(rac.)-(1R*, 5S*)-3-acetyl-7-{3-[2-(2,3,6-trifluorophenoxy)ethyl]phenyl}-3,9-diazabicyclo[3.3.1]non-6-ene-6-carboxylic acid methylphenethylamide;
(rac.)-(1R*, 5S*)-3-acetyl-7-{3-[2-(2,5-difluorophenoxy)ethyl]phenyl}-3,9-diazabicyclo[3.3.1]non-6-ene-6-carboxylic acid methylphenethylamide;
(rac.)-(1R*,5S*)-3-acetyl-7-[3-(2-o-tolyloxyethyl)phenyl]-3,9-diazabicyclo-[3.3.1]non-6-ene-6-carboxylic acid methylphenethylamide;
(rac.)-(1R*, 5S*)-3-acetyl-7-{3-[2-(2,3-dichlorophenoxy)ethyl]phenyl}-3,9-diazabicyclo[3.3.1]non-6-ene-6-carboxylic acid methylphenethylamide;
(rac.)-(1R*, 5S*)-3-acetyl-7-{3-[2-(2-chloro-5-methylphenoxy)ethyl]phenyl}-3,9-diazabicyclo[3.3.1]non-6-ene-6-carboxylic acid methylphenethylamide;
(rac.)-(1R*, 5S*)-3-acetyl-7-{3-[2-(3-chlorophenoxy)ethyl]phenyl}-3,9-diazabicyclo[3.3.1]non-6-ene-6-carboxylic acid methylphenethylamide;
(rac.)-(1R*, 5S*)-3-acetyl-7-{3-[3-(2-bromo-5-fluorophenoxy)propyl]phenyl}-3,9-diazabicyclo[3.3.1]non-6-ene-6-carboxylic acid methylphenethylamide;
(rac.)-(1R*, 5S*)-3-acetyl-7-{3-[2-(2-chlorophenoxy)propyl]phenyl}-3,9-diazabicyclo[3.3.1]non-6-ene-6-carboxylic acid methylphenethylamide;
(rac.)-(1R*, 5S*)-3-acetyl-7-{3-[2-(2-tert-butylphenoxy)propyl]phenyl}-3,9-diazabicyclo[3.3.1]non-6-ene-6-carboxylic acid methylphenethylamide;
(rac.)-(1R*, 5S*)-3-acetyl-7-{3-[2-(2,3,6-trifluorophenoxy)propyl]phenyl}-3,9-diazabicyclo[3.3.1]non-6-ene-6-carboxylic acid methylphenethylamide;
(rac.)-(1R*, 5S*)-3-acetyl-7-{3-[2-(2,5-difluorophenoxy)propyl]phenyl}-3,9-diazabicyclo[3.3.1]non-6-ene-6-carboxylic acid methylphenethylamide;
(rac.)-(1R*, 5S*)-3-acetyl-7-[3-(2-o-tolyloxypropyl)phenyl]-3,9-diazabicyclo-[3.3.1]non-6-ene-6-carboxylic acid methylphenethylamide;
(rac.)-(1R*, 5S*)-3-acetyl-7-{3-[2-(2,3-dichlorophenoxy)propyl]phenyl}-3,9-diazabicyclo[3.3.1]non-6-ene-6-carboxylic acid methylphenethylamide;
(rac.)-(1R*, 5S*)-3-acetyl-7-{3-[2-(2-chloro-5-methylphenoxy)propyl]phenyl}-3,9-diazabicyclo[3.3.1]non-6-ene-6-carboxylic acid methylphenethylamide;
(rac.)-(1R*, 5S*)-3-acetyl-7-{3-[2-(3-chlorophenoxy)propyl]phenyl}-3,9-diazabicyclo [3.3.1]non-6-ene-6-carboxylic acid methylphenethylamide;
(rac.)-(1R*, 5S*)-3-acetyl-7-{3-[2-(4-chlorophenoxy)propyl]phenyl}-3,9-diazabicyclo[3.3.1]non-6-ene-6-carboxylic acid methylphenethylamide;
(rac.)-(1R*, 5S*)-7-{3-[2-(2,6-dichloro-4-methylphenoxy)ethoxy]phenyl}-3,9-diazabicyclo[3.3.1]non-6-ene-6-carboxylic acid cyclopropyl-(3-methoxy-2-methylbenzyl)amide;
(rac.)-(1R*, 5S*)-7-{3-[4-(2-fluoro-3-trifluoromethylphenoxy)butoxy]phenyl}-3,9-diazabicyclo[3.3.1]non-6-ene-6-carboxylic acid cyclopropyl-(3-methoxy-2-methylbenzyl)amide;
(rac.)-(1R*, 5S*)-7-{3-[4-(2,6-dichloro-4-methylphenoxy)butoxy]phenyl}-3,9-diazabicyclo[3.3.1]non-6-ene-6-carboxylic acid cyclopropyl-(3-methoxy-2-methylbenzyl)amide; and
(rac.)-(1R*, 5S*)-7-{3-[4-(2-chloro-6-fluoro-3-methylphenoxy)butoxy]phenyl}-3,9-diazabicyclo[3.3.1]non-6-ene-6-carboxylic acid cyclopropyl-(3-methoxy-2-methylbenzyl)amide.

The compounds of general formula I and their pharmaceutically acceptable salts may be used as therapeutics e.g. in form of pharmaceutical compositions. These pharmaceutical compositions containing at least one compound of general formula I and usual carrier materials and adjuvants may especially be used in the treatment or prophylaxis of disorders which are associated with a dysregulation of the renin angiotensin system (RAS), comprising cardiovascular and renal diseases. Examples of such diseases are hypertension, congestive heart failure, pulmonary heart failure, coronary diseases, cardiac insufficiency, renal insufficiency, renal or myocardial ischemia, atherosclerosis, and renal failure. They can also be used to prevent restenosis after balloon or stent angioplasty, to treat erectile dysfunction, glomerulonephritis, renal colic, and glaucoma. Furthermore, they can be used in the therapy and the prophylaxis of diabetic complications, complications after vascular or cardiac surgery, complications of treatment with immunosuppresive agents after organ transplantation, complications of cyclosporin treatment, as well as other diseases presently known to be related to the RAS.

In another embodiment, the invention relates to a method for the treatment and/or prophylaxis of diseases which are related to the RAS comprising hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, complications of treatment with immunosuppressive agents after organ transplantation, and other diseases which are related to the RAS, which method comprises administering a compound according to general formula I to a human being or animal.

The invention further relates to the use of compounds of general formula I as defined above for the treatment and/or prophylaxis of disorders which are associated with the Renin Angiotensin System (RAS) comprising hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, complications of treatment with immunosuppressive agents after organ transplantation, and other diseases known to be related to the RAS.

In addition, the invention relates to the use of compounds as defined above for the preparation of medicaments for the treatment and/or prophylaxis of diseases which are associated with the RAS such as hypertension, coronary diseases, cardiac insufficiency, renal insufficiency, renal and myocardial ischemia, and renal failure.

The compounds of formula I may also be used in combination with one or more other pharmacologically active compounds comprising ACE inhibitors, angiotensin II receptor antagonists, endothelin receptor antagonists, vasodilators, calcium antagonists, potassium activators, diuretics, sympatholitics, beta-adrenergic antagonists, alpha-adrenergic antagonists, and neutral endopeptidase inhibitors, for the treatment of the above-mentioned diseases or disorders.

All forms of prodrugs leading to an active component comprised by general formula I above are included in the present invention.

The compounds of general formula I can be manufactured by the methods outlined below, by the methods described in the examples or by analogous methods.

Preparation of the Precursors:

Precursors are compounds which were prepared as key intermediates and/or building blocks and which were suitable for further transformations in parallel chemistry.

Bicyclononanone A was prepared from (4-benzyl-6-ethoxycarbonylmethyl-1-methylpiperazin-2-yl)acetic acid ethyl ester (Patent Application WO92/05174) as described in Scheme 1. Derivative A might also be present as enol form. In order to allow a coupling at the 7-position of bicyclononane A with aryl bromides, the vinyl triflate derivative B was prepared.

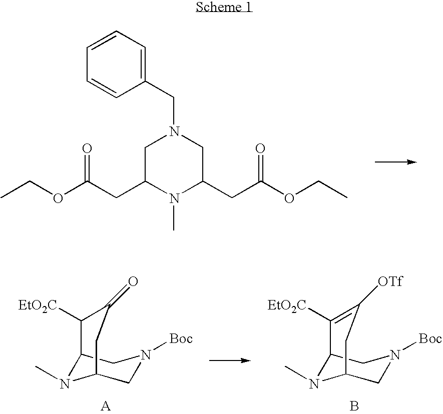

Compound B can be then transformed into compounds of type C by a Negishi coupling (Scheme 2), whereas R^{b} represents a side chain precursor suitable to construct the V—U-chain through one or several elementary chemical transformations. The R^{b}-substituent can be modified during the synthesis. After protecting group manipulations leading to compounds of type D the L¹-substituent can be put in place (compounds of type E), whereas L¹ represents a substituent L as defined in general formula I, or a precursor of such a substituent. The ester functional group can be saponified to compounds of type F. After an amide coupling for instance precursors of type G can be obtained.

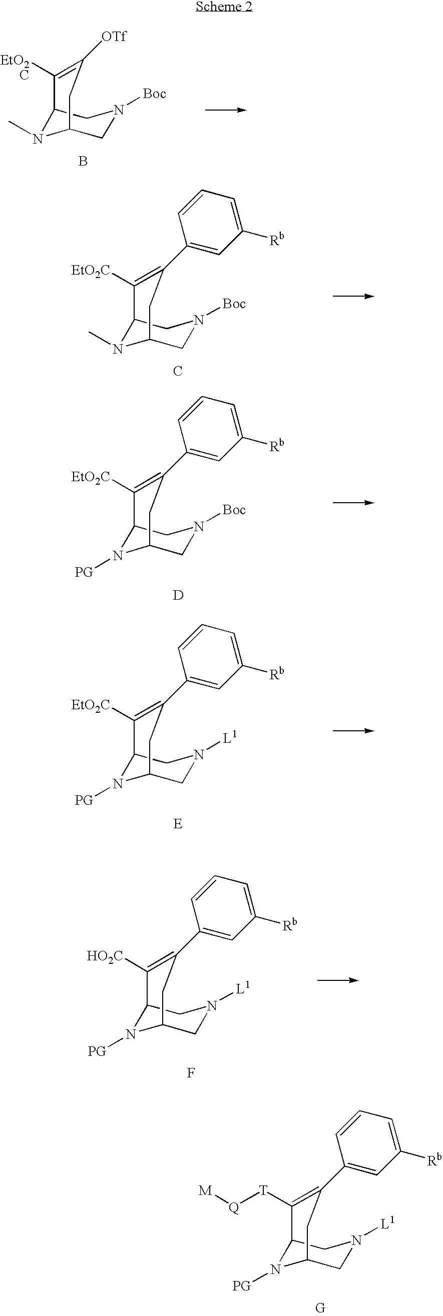

Other type of compounds described in the general formula I can be prepared using the chemistry described in patent application WO03/093267.

Otherwise heterocyclic systems may be prepared according to the literature existing for similar compounds. For instance pyridine derivative H could be prepared from 2,6-dibromopyridine, wherein R^{a} is a substituent that may be easily transformed into a chain U—V as described in Formula I (Scheme 3; Bitman, R., et al.; J. Org. Chem., 2000, 65, 7634). Pyridine J could be prepared by addition of a Grignard reagent on 4-bromopyridine, followed by oxidation (see Comins, D.; et al.; J. Org. Chem., 1985, 50, 4410). From known 3,5-dibromopyridine a compound of type K could be prepared. Finally pyridines of type L could be prepared from commercially available 2-(pyridin-4-yl)-alcohol, according to the literature described for similar compounds (Taylor, S. L.; et. al.; J. Org. Chem., 1983, 48, 4156).

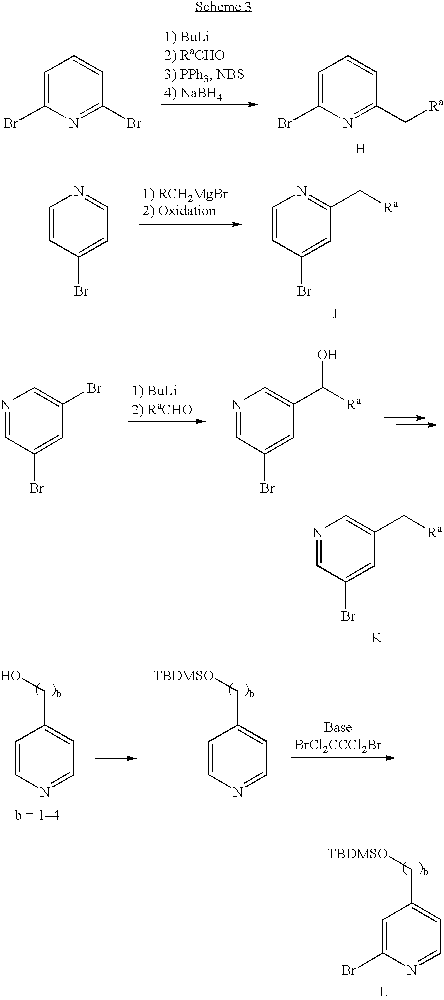

Pyridine derivatives of type M could be prepared according to the literature (Scheme 4, Newkome, G. R.; et al.; J. Am. Chem. Soc., 1975, 97, 3232). Known (4-bromopyridin-2-yloxy)acetic acid could lead to compound N by reduction. Compounds of type O could be prepared from 3,5-dibromopyridine, according to Harrowven, D. C.; et al.; Tetrahedron, 2001, 57, 4447. Known (2-bromopyridin-4-yl)acetic acid (Den Hertog, H. J.; et al.; Chem. Pharm. Bull, 1975, 23, 3008) could lead to compound P by reduction as well.

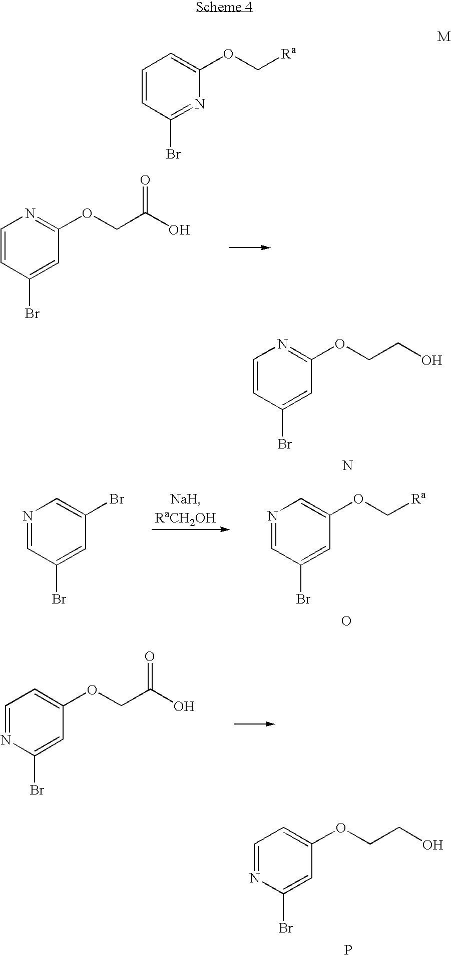

A reduction of the known (4-bromopyrimidin-2-yl)acetic acid methyl ester (Brown, D. J.; et al.; Australian J. Chem., 1978, 31, 649) could lead to the desired pyrimidine intermediate Q (Scheme 5). As well, a palladium-catalyzed coupling could transform 2,4-dibromopyrimidine into a compound of type R (see Gronowitz, S.; et al.; Chem. Scripta, 1986, 26, 305). Also, compounds of type S could be prepared from 6-alkyl-1H-pyrimidin-4-one.

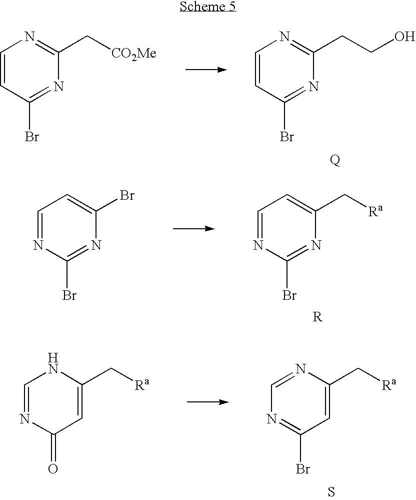

A palladium-catalized coupling of 2,4-dibromopyrimidine with bicyclononene B could lead to an intermediate of type T, which could be transformed into an intermediate of type U (Scheme 6). 6-Chloropyrazin-2-ylamine could be transformed in several steps into a pyrazine of type V (see Ghosh, A. K.; et al.; J. Med. Chem., 1993, 36, 2300 or Jovanovic, M. V., Heterocycles, 1983, 20, 2011, or Hartman, G. D.; et al.; J. Heteroclyclic Chem.; 1983, 20, 1089). Pyrimidines of type W could be prepared according to standard procedures. Finally a compounds of type X can be prepared from 4,6-dichloropyrimidine.

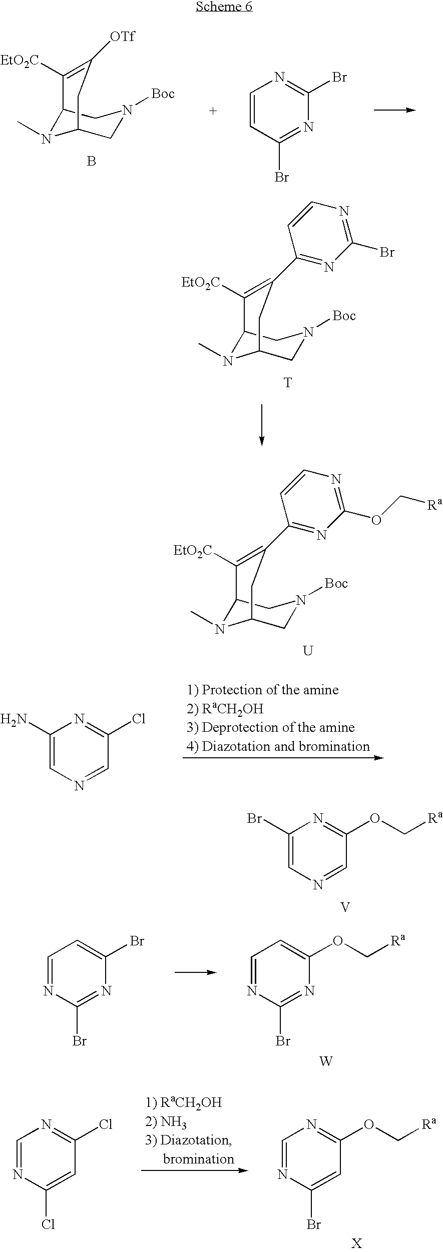

Pyridazinyl derivatives may be prepared as described in Scheme 7. For instance known 2-pyridazin-3-ylethanol Y (Rodriguez, L.; et al.; Synlett, 1990, 227) could be transformed into a pyridazinyl derivative Z following known chemistry (Sauer, J.; et al.; Tetrahedron, 1998, 54, 4297). Known 6-bromo-pyridazine-4-carboxylic acid ethyl ester AA (Dulayyi, A.; et al.; Tetrahedron, 1998, 54, 12897; Barlin, G. B.; et al.; Australian J. Chem., 1977, 30, 2319) could be alkylated, then reduced to a derivative AB bearing the desired R^{a}-substituent. Also, known compound AC (Brundish, D. E.; et al.; J. Labelled Compounds and Radiopharmaceuticals, 1988, 25, 1371) could be transformed to derivative AD that could be reduced to pyridazinyl derivative AE. Hydrolysis could lead to derivative AF, finally O-alkylation to the desired intermediate AG. 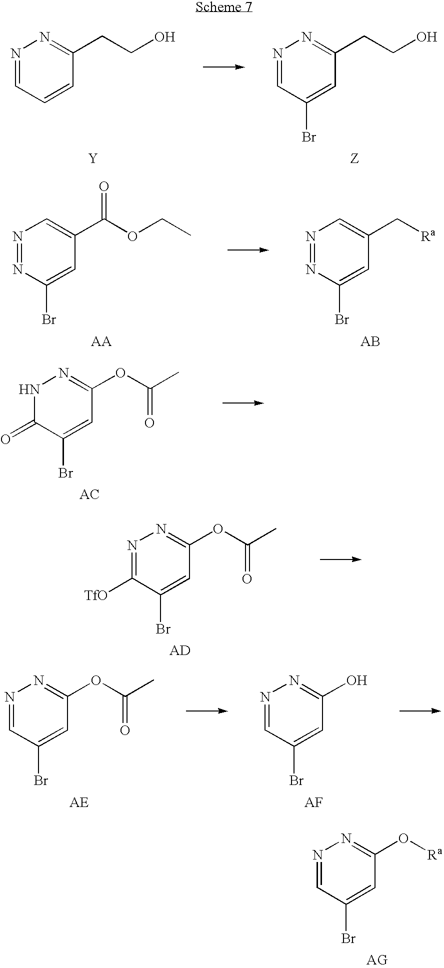
Preparation of Final Compounds

Precursors G were transformed into the corresponding aryl ethers (Scheme 8), using the Mitsunobu reaction conditions. After deprotection; the final compounds are obtained.

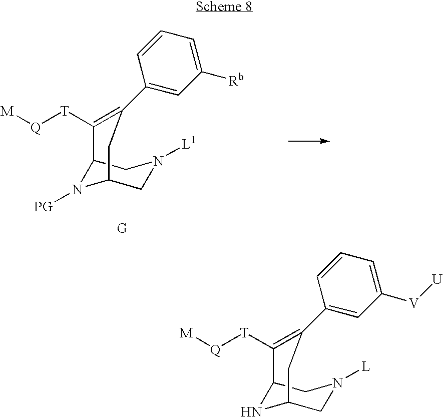

The compounds of formula I and their pharmaceutically acceptable acid addition salts can be used as medicaments, e.g. in the form of pharmaceutical preparations for enteral, parenteral, or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils.

The production of pharmaceutical preparations can be effected in a manner which will be familiar to any person skilled in the art by bringing the described compounds of formula I and their pharmaceutically acceptable acid addition salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers are, however, required in the case of soft gelatine capsules). Suitable carrier materials for the production of solutions and syrups are, for example, water, polyols, sucrose, invert sugar and the like. Suitable carrier materials for injections are, for example, water, alcohols, polyols, glycerols and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of compounds of formula I can vary within wide limits depending on the disease to be controlled, the age and the individual condition of the patient and the mode of administration, and will, of course, be fitted to the individual requirements in each particular case. For adult patients a daily dosage of about 1 mg to about 1000 mg, especially about 50 mg to about 500 mg, comes into consideration.

The pharmaceutical preparations conveniently contain about 1-500 mg, preferably 5-200 mg of a compound of formula I.

The following examples serve to illustrate the present invention in more detail. They are, however, not intended to limit its scope in any manner.

### EXAMPLES

General Remarks

The compounds were characterized at least by LC-MS and ¹H-NMR. Only the LC-MS data are given here.

Abbreviations

AcCl Acetyl chloride

ACE Angiotensin Converting Enzyme

AcOH Acetic acid

Ang Angiotensin

aq. aqueous

Bn Benzyl

Boc tert-Butyloxycarbonyl

BSA Bovine serum albumine

BuLi n-Butyllithium

DIPEA Diisopropylethylamine

DMAP 4-N,N-Dimethylaminopyridine

DMSO Dimethylsulfoxide

EDC.HCl Ethyl-N,N-dimethylaminopropylcarbodiimide hydrochloride

EIA Enzyme immunoassay

eq. equivalent

Et Ethyl

EtOAc Ethyl acetate

FC Flash Chromatography

HOBt Hydroxybenzotriazol

MeOH Methanol

org. organic

PG protecting group

Ph Phenyl

RAS Renin Angiotensin System

RP18 Reversed phase column, filled with C₁₈ hydrocarbon

rt room temperature

sol. Solution

TBAF Tetrabutylammonium fluoride

TBDMS tert-Butyldimethylsilyl

tBuOK Potassium tert-butylate

Tf Trifluoromethylsulfonyl

TFA Trifluoroacetic acid

THF Tetrahydrofuran

TLC Thin Layer Chromatography

TMAD N,N,N′,N′-Tetramethylazodicarboxamide

Preparation of the Precursors

### [4-(3-Bromophenoxy)butoxy]-tert-butyldimethylsilane

A mixture of 3-bromophenol (10.0 g, 56.6 mmol), KI (50 mg) and K₂CO₃ (12.5 g, 90.6 mmol) in DMF (100 mL) was stirred at 85° C. for 30 min. 4-Bromobutanol (8.75 g, 57.2 mmol) was added and the mixture was stirred at 85° C. for 16 h. 4-Bromobutanol (8.75 g, 57.2 mmol) was added again and the mixture was stirred at 85° C. overnight. The mixture was poured onto a mixture of ice and water (500 mL), and extracted with CH₂Cl₂. The combined org. extracts were washed with aq. 1M NaOH, dried over MgSO₄, filtered, and the solvents were removed under reduced pressure. Purification by FC yielded 4-(3-bromophenoxy)-butan-1-ol (4.66 g, 34%).

This compound was dissolved in DMF (75 mL), and imidazol (3.27 g, 47.5 mmol), then TBDMS-Cl (3.02 g, 19.0 mmol) were added. The mixture was stirred at rt overnight. The mixture was poured onto a sol. of NaHCO₃ (0.5 g) in water (125 mL). The resulting mixture was extracted with Et₂O (2×). The combined org. extracts were dried over MgSO₄, filtered, and the solvents were removed under reduced pressure. Purification by FC yielded the title compound (6.78 g, quantitative yield).

### (rac.)-(1R*, 5S*)-9-Methyl-7-oxo-3,9-diazabicyclo[3.3.1]nonane-3,6-dicarboxylic acid 3-tert-butyl ester 6-ethyl ester (A)

(4-Benzyl-6-ethoxycarbonylmethyl-1-methyl-piperazin-2-yl)acetic acid ethyl ester (Patent WO 92/05174) (71.0 g, 0.196 mol) was dissolved in MeOH (400 mL). TFA (77.8 mL, 1.02 mol) was added and the flask was purged with nitrogen. Pd/C (10%, 50% moisture, 3.6 g) was added. The flask was closed and purged with hydrogen (3×). After 1 day, the mixture was filtered through Celite and washed with MeOH. The solvents were removed under reduced pressure and the foamy residue (92.7 g) was dried under high vacuum. A sol. of tBuOK (117.2 g, 1.04 mol) in toluene (3.07 L) was heated to reflux. A sol. of the crude piperazine formerly obtained, dissolved in THF (300 mL), was added dropwise over 50 min. The black mixture was stirred for 10 further min. and allowed to cool to rt. The mixture was cooled to 0° C. and AcOH (36.6 mL, 0.635 mol) was added. The solvents were removed under reduced pressure. This crude material was suspended in CH₂Cl₂ (400 mL) and cooled to 0° C. DIPEA (19.1 mL, 112 mmol) was added. A sol. of Boc₂O (24.3 g, 113 mmol) in CH₂Cl₂ (200 mL) was added dropwise. The mixture was stirred for 1 h at 0° C., then 1 h at rt. The mixture was washed with aq. 10% Na₂CO₃ (2×). The org. extracts were dried over MgSO₄, filtered and the solvents were evaporated under reduced pressure. The residue was purified by FC (EtOAc/heptane 1:1→EtOAc). The title compound was obtained as oil (24.5 g, 38%). R_{f}=0.05 (EtOAc/heptane 1:1) or 0.56 (MeOH/CH₂Cl₂ 1:9). LC-MS: Rₜ=2.94; ES+: 325.19.

### (rac.)-(1R*, 5S*)-9-Methyl-7-trifluoromethanesulfonyloxy-3,9-diazabicyclo-[3.3.1]non-6-ene-3,6-dicarboxylic acid 3-tert-butyl ester 6-ethyl ester (B)

A sol. of bicyclononanone A (2.22 g, 6.80 mmol) in THF (50 mL) was cooled to 0° C. and NaH (about 60% in mineral oil, 326 mg, about 8.2 mmol) was added. A gas evolution was observed. After 20 min, Tf₂NPh (3.22 g, 9.00 mmol) was added. 10 min later, the ice bath was removed. After 3 h, the sol. was diluted with EtOAc and washed with brine (1×). The org. extracts were dried over MgSO₄, filtered, and the solvents were removed under reduced pressure. Purification by FC (EtOAc/heptane 3:1→EtOAc) yielded the title compound as an oil (2.50 g, 80%). R_{f}=0.15 (EtOAc/heptane 1:1). LC-MS: Rₜ=4.73; ES+: 458.95.

### (rac.)-(1R*, 5S*)-7-{3-[2-(tert-Butyldimethylsilanyloxy)ethyl]phenyl}-9-methyl-3,9-diazabicyclo[3.3.1]non-6-ene-3,6-dicarboxylic acid 3-tert-butyl ester 6-ethyl ester (C1)

BuLi (1.6M in hexane, 8.52 mL, 13.6 mmol) was added to a sol. of [2-(3-bromophenyl)ethoxy]-tert-butyldimethylsilane (4.28 g, 13.6 mmol, prepared by silylation of 2-(3-bromophenyl)ethanol) in THF (60 mL) at −78° C. The mixture was stirred for 30 min at this temperature, and ZnCl₂ (1M in THF, prepared from ZnCl₂ dried at 160° C. for 3 h and THF, 15.9 mL, 15.9 mmol) was added. The mixture was allowed to warm to rt and a sol. of bicyclononene B (2.50 g, 5.45 mmol) in THF (15 ml), then Pd(PPh₃)₄ (147 mg, 0.13 mmol) were added. The mixture was heated to 40° C. and stirred at this temperature for 30 min. The mixture was allowed to cool to rt and aq. 1M HCl (2 mL) was added. The mixture was diluted with EtOAc and washed with aq. 1M NaOH (1×). The org. extracts were dried over MgSO₄, filtered, and the solvents were removed under reduced pressure. Purification by FC (MeOH/CH₂Cl₂ 10:490→15:485→20:480→25:475→50:450) yielded the title compound (2.41 g, 80%). LC-MS; Rₜ=5.02 ES+: 545.31.

### (rac.)-(1R*, 5S*)-7-{3-[2-(tert-Butyldimethylsilanyloxy)propyl]phenyl}-9-methyl-3,9-diazabicyclo[3.3.1]non-6-ene-3,6-dicarboxylic acid 3-tert-butyl ester 6-ethyl ester (C2)

BuLi (1.6M in hexane, 10.22 mL, 16.4 mmol) was added to a sol. of [2-(3-bromophenyl)propoxy]-tert-butyldimethylsilane (4.95 g, 16.3 mmol, prepared by silylation of 2-(3-bromophenyl)propanol, P. Beak, et al., J. Org. Chem., 1989, 54, 5574) in THF (60 mL) at −78° C. The mixture was stirred for 30 min at this temperature, and ZnCl₂ (1M in THF, prepared from ZnCl₂ dried at 160° C. for 3 h and THF, 19.0 mL, 19.0 mmol) was added. The mixture was allowed to warm to rt and a sol. of bicyclononene B (3.00 g, 6.54 mmol) in THF (15 ml), then Pd(PPh₃)₄ (189 mg, 0.16 mmol) were added. The mixture was heated to 40° C. and stirred at this temperature for 30 min. The mixture was allowed to cool to rt and aq. 1M HCl (2 mL) was added. The mixture was diluted with EtOAc and washed with aq. 1M NaOH (1×). The org. extracts were dried over MgSO₄, filtered, and the solvents were removed under reduced pressure. Purification by FC (MeOH/CH₂Cl₂ 10:490→15:485→20:480→25:475→50:450) yielded the title compound (2.74 g, 75%). LC-MS; Rₜ=5.10 ES+: 559.32.

### (rac.)-(1R*, 5S*)-7-{3-[2-(tert-Butyldimethylsilanyloxy)ethoxy]phenyl}-9-methyl-3,9-diazabicyclo[3.3.1]non-6-ene-3,6-dicarboxylic acid 3-tert-butyl ester 6-ethyl ester (C3)

BuLi (1.6M in hexane, 19.2 mL, 30.7 mmol) was added to a sol. of [2-(4-bromo-phenoxy)ethoxy]-tert-butyldimethylsilane (9.40 g, 28.4 mmol, Morita, C.; et al, Heterocycles, 2000, 52, 1163) in THF (55 mL) at −78° C. The mixture was stirred for 30 min at this temperature, and ZnCl₂ (1M in THF, prepared from ZnCl₂ dried at 160° C. for 3 h and THF, 35.5 mL, 35.5 mmol) was added. The mixture was allowed to warn to rt and a sol. of bicyclononene B (10.8 g, 23.6 mmol) in THF (20 ml), then Pd(PPh₃)₄ (0.70 g, 0.59 mmol) were added. The mixture was heated to 40° C. and stirred at this temperature for 30 min. The mixture was allowed to cool to rt and aq. 1M HCl (2 mL) was added. The mixture was diluted with EtOAc and washed with aq. 1M NaOH (1×). The org. extracts were dried over MgSO₄, filtered, and the solvents were removed under reduced pressure. Purification by FC (MeOH/CH₂Cl₂ 10:490→15:485→20:480→25:475→50:450) yielded the title compound (11.7 g, 89%). LC-MS; Rₜ=0.98 ES+: 561.10.

### (rac.)-(1R*, 5S*)-7-{3-[4-(tert-Butyldimethylsilanyloxy)butoxy]phenyl}-9-methyl-3,9-diazabicyclo[3.3.1]non-6-ene-3,6-dicarboxylic acid 3-tert-butyl ester 6-ethyl ester (C4)

BuLi (1.6M in hexane, 12.75 mL, 20.4 mmol) was added to a sol. of [4-(3-bromophenoxy)butoxy]-tert-butyldimethylsilane (6.78 g, 18.8 mmol) in THF (100 mL) at −78° C. The mixture was stirred for 30 min at this temperature, and ZnCl₂ (1M in THF, prepared from ZnCl₂ dried at 160° C. for 3 h and THF, 23.6 mL, 23.6 mmol) was added. The mixture was allowed to warm to rt and a sol. of bicyclononene B (7.2 g, 15.7 mmol) in THF (20 ml), then Pd(PPh₃)₄ (0.468 g, 0.393 mmol) were added. The mixture was stirred at rt for 1 h. The mixture was allowed to cool to rt and aq. 1M HCl (2 mL) was added. The mixture was diluted with EtOAc and washed with aq. 1M NaOH (1×). The org. extracts were dried over MgSO₄, filtered, and the solvents were removed under reduced pressure. Purification by FC (MeOH/CH₂Cl₂ 10:490→15:485→20:480→25:475→50:450) yielded the title compound (2.60 g, 28%). LC-MS; Rₜ=1.02 ES+: 589.10.

### (rac.)-(1R*, 5S*)-7-{3-[2-(tert-Butyldimethylsilanyloxy)ethyl]phenyl}-3,9-diazabicyclo[3.3.1]non-6-ene-3,6,9-tricarboxylic acid 3-tert-butyl ester 6-ethyl ester 9-(2,2,2-trichloro-1,1-dimethylethyl)ester (D1)

A sol. of bicyclononene C1 (2.3 g, 4.22 mmol) and β,β,β-trichloro-tert-butylchloroformate (10.4 g, 43.3 mmol) in CH₂ClCH₂Cl (40 ml) was heated to reflux for 3 h. The mixture was allowed to cool to rt and the solvents were removed under reduced pressure. Purification of the residue by FC (EtOAc/heptane 1:9→1:4→2:3) yielded the title compound (2.73 g, 88%). LC-MS: Rₜ=8.00 min.

### (rac.)-(1R*, 5S*)-7-{3-[2-(tert-Butyldimethylsilanyloxy)propyl]phenyl}-3,9-diazabicyclo[3.3.1]non-6-ene-3,6,9-tricarboxylic acid 3-tert-butyl ester 6-ethyl ester 9-(2,2,2-trichloro-1,1-dimethylethyl)ester (D2)

A sol. of bicyclononene C2 (2.74 g, 4.90 mmol) and β,β,β-trichloro-tert-butylchloroformate (11.76 g, 49.0 mmol) in CH₂ClCH₂Cl (50 ml) was heated to reflux for 3 h. The mixture was allowed to cool to rt and the solvents were removed under reduced pressure. Purification of the residue by FC (EtOAc/heptane 1:9→1:4→2:3) yielded the title compound (3.01 g, 85%). LC-MS: Rₜ=8.05 min.

### (rac.)-(1R*, 5S*)-7-[3-(2-Hydroxyethoxy)phenyl]-3,9-diazabicyclo[3.3.1]non-6-ene-3,6,9-tricarboxylic acid 3,9-di-tert-butyl ester 6-ethyl ester (D3)

NaHCO₃ (17.4 g, 208 mmol) and 1-chloroethyl chloroformate (22.4 mL, 208 mmol) were added to a sol. of bicyclononene C3 (11.65 g, 20.8 mmol) in 1,2-dichloroethane (180 mL). The sol. was heated to reflux. After 4.5 h, the reaction mixture was allowed to cool to rt, and the solvents were removed under reduced pressure. MeOH (180 mL) was added. The mixture was stirred at rt for 30 min, and the solvents were removed under reduced pressure. The residue was dissoled in CH₂Cl₂ (220 mL), DIPEA (17.8 mL, 104 mmol) was added, and the mixture was cooled to 0° C. Boc₂O (13.6 g, 62.4 mmol) was added and the mixture was stirred at rt for 2 h. The mixture was washed with aq. 1M HCl (1×), and aq. sat. NaHCO₃ (1×). The org. extracts were dried over MgSO₄, filtered, and the solvents were removed under reduced pressure. Purification of the residue by FC yielded the title compound (9.57 g, 87%). LC-MS: Rₜ=1.02 min; ES+: 477.02.

### (rac.)-(1R*, 5S*)-7-[3-(4-Hydroxybutoxy)phenyl]-3,9-diazabicyclo[3.3.1]non-6-ene-3,6,9-tricarboxylic acid 3,9-di-tert-butyl ester 6-ethyl ester (D4)

NaHCO₃ (3.74 g, 44.2 mmol) and 1-chloroethyl chloroformate (4.85 mL, 44.2 mmol) were added to a sol. of bicyclononene C4 (2.60 g, 4.41 mmol) in 1,2-dichloroethane (30 mL). The sol. was heated to reflux. After 3 h, the reaction mixture was allowed to cool to rt, and the solvents were removed under reduced pressure. MeOH (30 mL) was added. The mixture was stirred at rt for 30 min, and the solvents were removed under reduced pressure. The residue was dissoled in CH₂Cl₂ (50 mL), DIPEA (3.75 mL, 22.1 mmol) was added, and the mixture was cooled to 0° C. Boc₂O (2.95 g, 13.2 mmol) was added and the mixture was stirred at rt for 2 h. The mixture was washed with aq. 1M HCl (1×), and aq. sat. NaHCO₃ (1×). The org. extracts were dried over MgSO₄, filtered, and the solvents were removed under reduced pressure. Purification of the residue by FC yielded the title compound (1.71 g, 69%). LC-MS: Rₜ=1.05 min; ES+: 505.08.

### (rac.)-(1R*, 5S*)-3-Acetyl-7-[3-(2-hydroxyethyl)phenyl]-3,9-diazabicyclo-[3.3.1]non-6-ene-6,9-dicarboxylic acid 6-ethyl ester 9-(2,2,2-trichloro-1,1-dimethylethyl)ester (E1)

HCl (4M in dioxane, 30 mL) was added to a sol. of bicyclononene D1 (2.73 g, 3.72 mmol) in CH₂Cl₂ (30 ml) at 0° C. The mixture was stirred at 0° C. for 20 min. then at rt for 2.5 h. The solvents were evaporated under reduced pressure and the residue was dried under high vacuum. A mixture of this residue, DMAP (23 mg, 0.19 mmol,) and DIPEA (2.54 ml, 15.3 mmol) in THF (15 mL) was cooled to −78° C. AcCl (0.29 mL, 3.70 mmol) was added slowly. The mixture was stirred for 30 min, and MeOH (2 mL) was added. The mixture was allowed to warm up to rt and was diluted with EtOAc, washed with aq. 1M HCl (2×) and aq. sat. NaHCO₃ (1×). The org. extracts were dried over MgSO₄, filtered, and the solvents were removed under reduced pressure. Purification of the residue by FC (EtOAc/heptane 1:1→EtOAc→EtOAc/MeOH 1:9) yielded the title compound (1.25 g, 60%). LC-MS: Rₜ=6.62.

### (rac.)-(1R*, 5S*)-3-Acetyl-7-[3-(2-hydroxypropyl)phenyl]-3,9-diazabicyclo-[3.3.1]non-6-ene-6,9-dicarboxylic acid 6-ethyl ester 9-(2,2,2-trichloro-1,1-dimethylethyl)ester (E2)

HCl (4M in dioxane, 30 mL) was added to a sol. of bicyclononene D2 (3.01 g, 4.02 mmol) in CH₂Cl₂ (30 ml) at 0° C. The mixture was stirred at 0° C. for 20 min, then at rt for 2.5 h. The solvents were evaporated under reduced pressure and the residue was dried under high vacuum. A mixture of this residue, DMAP (25 mg, 0.20 mmol,) and DIPEA (2.75 ml, 16 mmol) in THF (20 mL) was cooled to −78° C. AcCl (0.31 mL, 4.10 mmol) was added slowly. The mixture was stirred for 30 min, and MeOH (2 mL) was added. The mixture was allowed to warm up to rt and was diluted with EtOAc, washed with aq. 1M HCl (2×) and aq. sat. NaHCO₃ (1×). The org. extracts were dried over MgSO₄, filtered, and the solvents were removed under reduced pressure. Purification of the residue by FC (EtOAc/heptane 1:1→EtOAc→EtOAc/MeOH 1:9) yielded the title compound (1.56 g, 69%).

### (rac.)-(1R*, 5S*)-3-Acetyl-7-[3-(2-hydroxyethyl)phenyl]-3,9-diazabicyclo[3.3.1]non-6-ene-6,9-dicarboxylic acid 9-(2,2,2-trichloro-1,1-dimethylethyl) ester (F1)

A mixture of bicyclononene E1 (1.25 g, 2.22 mmol) in EtOH (56 ml) and aq. 1M NaOH (56 mL) was stirred at 80° C. for 2.5 h. The mixture was allowed to cool to rt and acidified with aq 1M HCl. The mixture was extracted with EtOAc (3×) and the org. extracts were dried over MgSO₄, filtered, and the solvents were removed under reduced pressure. Purification of the crude by FC (EtOAc→MeOH/EtOAc 1:19→1:9) yielded the title compound (360 mg 35%). LC-MS: Rₜ=4.66; MS−: 530.82.

### (rac.)-(1R*, 5S*)-3-Acetyl-7-[3-(2-hydroxypropyl)phenyl]-3,9-diazabicyclo[3.3.1]non-6-ene-6,9-dicarboxylic acid 9-(2,2,2-trichloro-1,1-dimethylethyl) ester (F2)

A mixture of bicyclononene E2 (1.56 g, 2.70 mmol) in EtOH (70 ml) and aq. 1M NaOH (70 mL) was stirred at 80° C. for 2.5 h. The mixture was allowed to cool to rt and acidified with aq 1M HCl. The mixture was extracted with EtOAc (3×) and the org. extracts were dried over MgSO₄, filtered, and the solvents were removed under reduced pressure. Purification of the crude by FC (EtOAc→MeOH/EtOAc 1:19→1:9) yielded the title compound (1.18 g 80%).

### (rac.)-(1R*, 5S*)-7-[3-(tert-Butyldimethylsilanyloxy)ethoxyphenyl]-3,9-diazabicyclo[3.3.1]non-6-ene-3,6,9-tricarboxylic acid 3,9-di-tert-butyl ester (F3)

A mixture of bicyclononene D3 (6.27 g, 11.8 mmol) in EtOH (210 ml) and aq. 1M NaOH (90 mL) was stirred at 80° C. for 5.5 h. The mixture was allowed to cool to rt and acidified with aq 1M HCl. The mixture was extracted with EtOAc (3×) and the org. extracts were dried over MgSO₄, filtered, and the solvents were removed under reduced pressure. The crude compound (5.21 g 10.4 mmol), imidazol (2.12 g, 31.2 nmol) and TBDMS-Cl (3.45 g, 22.9 mmol) in DMF (50 mL) were stirred at rt overnight. Aq. sat. NH₄Cl was added and the mixture was extracted with hexane (3×). The combined org. extracts were dried over MgSO₄, filtered, and the solvents were evaporated under reduced pressure. A mixture of this crude product and K₂CO₃ (0.6 g) in THF (30 ml), MeOH (10 ml), and H₂O (10 ml) was stirred at rt for 3 h. Aq. sat. NH₄Cl was added and this mixture was extracted with Et₂O (3×). The combined org. extracts were dried over MgSO₄, filtered, and the solvents were removed under reduced pressure. This yielded the title compound (6.21 g, 85%) that was used withour further purification.

### (rac.)-(1R*, 5S*)-7-{3-[4-(tert-Butyldimethylsilanyloxy)butoxy]phenyl}-3,9 diazabicyclo[3.3.1]non-6-ene-3,6,9-tricarboxylic acid 3,9-di-tert-butyl ester (F4)

A mixture of bicyclononene D4 (1.71 g, 3.05 mmol) in EtOH (35 ml) and aq. 1M NaOH (15 mL) was stirred at 80° C. for 4 h. The mixture was allowed to cool to rt and acidified with aq 1M HCl. The mixture was extracted with EtOAc (3×) and the org. extracts were dried over MgSO₄, filtered, and the solvents were removed under reduced pressure. The crude compound (1.65 g 3.05 mmol), imidazol (0.85 g, 12.4 mmol) and TBDMS-Cl (2.50 g, 7.7 mmol) in DMF (35 mL) were stirred at rt overnight. Aq. sat. NH₄Cl was added and the mixture was extracted with hexane (3×). The combined org. extracts were dried over MgSO₄, filtered, and the solvents were evaporated under reduced pressure. A mixture of this crude product and K₂CO₃ (0.6 g) in THF (30 ml), MeOH (10 ml), and H₂O (10 ml) was stirred at rt for 3 h. Aq. sat NH₄Cl was added and this mixture was extracted with Et₂O (3×). The combined org. extracts were dried over MgSO₄, filtered, and the solvents were removed under reduced pressure. This yielded the title compound (1.79 g, 87%) that was used withour further purification. LC-MS: Rₜ=1.35; ES+: 783.31.

### (rac.)-(1R*, 5S*)-3-Acetyl-7-[3-(2-hydroxyethyl)phenyl]-6-(methyl-phenethyl-carbamoyl)-3,9-diazabicyclo[3.3.1]non-6-ene-9-carboxylic acid 2,2,2-trichloro-1,1-dimethylethyl ester (G1)

A mixture of bicyclononene F1 (360 mg, 0.67 mmol), methylphenethylamine (0.28 mL, 2.01 mmol), DMAP (20 mg, 0.17 mmol), DIPEA (0.74 mL, 4.30 mmol), HOBt (22 mg, 0.16 mmol) and EDC.HCl (310 mg, 1.68 mmol) in CHCl₃ (10 mL) was stirred for 3 days. The mixture was diluted in CH₂Cl₂ and washed with aq. 1M HCl (2×), and aq. sat. NaHCO₃ (1×). The org. extracts were dried over MgSO₄, filtered, and the solvents were removed under reduced pressure. Purification by FC (EtOAc/heptane 1:4→1:1→4:1→EtOAc→MeOH/EtOAc 1:19→90:10) yielded the title compound (350 mg, 80%). LC-MS: Rₜ=5.37; ES+: 650.19.

### (rac.)-(1R*, 5S*)-3-Acetyl-7-[3-(2-hydroxypropyl)phenyl]-6-(methylphenethylcarbamoyl)-3,9-diazabicyclo[3.3.1]non-6-ene-9-carboxylic acid 2,2,2-trichloro-1,1-dimethylethyl ester (G2)

A mixture of bicyclononene E2 (790 mg, 1.44 mmol), methylphenethylamine (0.63 mL, 4.33 mmol), DMAP (44 mg, 0.36 mmol), DIPEA (0.99 mL, 5.94 mmol), HOBt (48 mg, 0.36 mmol) and EDC.HCl (690 mg, 3.60 mmol) in CHCl₃ (20 mL) was stirred for 3 days. The mixture was diluted in CH₂Cl₂ and washed with aq. 1M HCl (2×), and aq. sat. NaHCO₃ (1×). The org. extracts were dried over MgSO₄, filtered, and the solvents were removed under reduced pressure. Purification by FC (EtOAc/heptane 1:4→1:1→4:1→EtOAc→MeOH/EtOAc 1:19→90:10) yielded the title compound (750 mg, 78%). LC-MS: Rₜ=5.43; ES+: 664.13.

### (rac.)-(1R*, 5S*)-6-[Cyclopropyl-(3-methoxy-2-methylbenzyl)carbamoyl]-7-[3-(2-hydroxyethoxy)phenyl]-3,9-diazabicyclo[3.3.1]non-6-ene-3,9-dicarboxylic acid di-tert-butyl ester (G3)

(1-Chloro-2-methylpropenyl)dimethylamine (1.50 mL, 11.6 mmol) was added to a sol. of compound F3 (6.5 g, 10.5 mmol) in CH₂Cl₂ (100 mL) at rt. The mixture was stirred for 20 min. Cyclopropyl-(3-methoxy-2-methylbenzyl)amine (prepared by reductive amination from 3-methoxy-2-methylbenzaldehyde, Comins, D. L.; Brown, J. D., J. Org. Chem., 1989, 54, 3730 and cyclopropylamine, 2.01 g, 10.5 mmol), and DIPEA (11.0 mL, 31.5 mmol) were added. The mixture was stirred for 35 min, and aq. 10% citric acid was added. The phases were separated, and the org. phase was washed with aq. 10% citric acid. The combined aq. phases were extracted back with CH₂Cl₂. The combined org. extracts were dried over MgSO₄, filtered, and the solvents were removed under reduced pressure. The crude title compound (8.30 g, 10.5 mmol) was dissolved in THF (170 mL) and TBAF (3.70 g, 11.5 mmol) was added. The mixture was stirred for 25 min. The solvents were removed under reduced pressure, and the residue was diluted with EtOAc. The mixture was washed with water, and brine. The org. extracts were dried over MgSO₄, filtered, and the solvents were removed under reduced pressure. Purification of the residue by FC yielded the title compound (4.69 g, 66%). LC-MS: Rₜ=1.07; ES+: 678.32.

### (rac.)-(1R*, 5S*)-6-[Cyclopropyl-(3-methoxy-2-methylbenzyl)carbamoyl]-7-[3-(4-hydroxybutoxy)phenyl]-3,9-diazabicyclo[3.3.1]non-6-ene-3,9-dicarboxylic acid di-tert-butyl ester (G4)

(1-Chloro-2-methylpropenyl)dimethylamine (0.42 mL, 3.04 mmol) was added to a sol. of compound F4 (1.79 g, 2.76 mmol) in CH₂Cl₂ (50 mL) at rt. The mixture was stirred for 30 min. Cyclopropyl-(3-methoxy-2-methylbenzyl)amine (prepared by reductive amination from 3-methoxy-2-methylbenzaldehyde, Comins, D. L.; Brown, J. D., J. Org. Chem., 1989, 54, 3730 and cyclopropylamine, 0.53 g, 2.76 mmol), and DIPEA (1.44 mL, 8.30 mmol) were added. The mixture was stirred for 1 h, and aq. 10% citric acid was added. The phases were separated, and the org. phase was washed with aq. 10% citric acid. The combined aq. phases were extracted back with CH₂Cl₂. The combined org. extracts were dried over MgSO₄, filtered, and the solvents were removed under reduced pressure. The crude title compound (2.29 g) was dissolved in THF (50 mL) and TBAF (1.82 g, 5.58 mmol) was added. The mixture was stirred for 2 h. The solvents were removed under reduced pressure, and the residue was diluted with EtOAc. The mixture was washed with water, and brine. The org. extracts were dried over MgSO₄, filtered, and the solvents were removed under reduced pressure. Purification of the residue by FC yielded the title compound (1.22 g, 62%). LC-MS: Rₜ=1.09; ES+: 706.28.

Preparation of the Final Compounds

General Procedure (A) for the Formation of Aryl Ether (Mitsunobu Reaction)

The precursor (0.05 mmol) was dissolved or suspended in toluene (1.00 mL). The phenol derivative (0.075 mmol) in toluene (0.50 mL) was added. TMAD (0.075 mmol) in toluene (0.50 mL) was added, followed by tributylphosphine (0.15 mmol). The reaction mixture was stirred for 2 h at rt and then 2 h at 60° C. Sometimes, it was necessary to add a second portion of tributylphosphine and to stir overnight. Sometimes, THF was necessary as cosolvent to dissolve the reactants. The reaction mixture was allowed to cool to rt, then water was added. The mixture was extracted with EtOAc, and the org. extracts were evaporated under reduced pressure.

General Procedure (B) for the Cleavage of the 2,2,2-trichloro-1,1-dimethylethylcarbamate Protecting Group:

The crude product from another general procedure was dissolved in THF/AcOH (1:0.1) and treated with zinc (20 eq.). The suspension was stirred for 5 h and filtered through celite, which was washed with EtOAc. The filtrate was evaporated under reduced pressure and the residue was purified by HPLC.

### EXAMPLES

### Example 1

### (rac.)-(1R*, 5S*)-3-Acetyl-7-{3-[2-(2-chlorophenoxy)ethyl]phenyl}-3,9-diazabicyclo[3.3.1]non-6-ene-6-carboxylic acid methylphenethylamide formate salt

According to the general procedures A and B, starting from compound G1 and 2-chlorophenol. LC-MS: Rₜ=0.83 min, ES+=558.11.

### Example 2

### (rac.)-(1R*, 5S*)-3-Acetyl-7-{3-[2-(2-tert-butylphenoxy)ethyl]phenyl}-3,9-diazabicyclo[3.3.1]non-6-ene-6-carboxylic acid methylphenethylamide formate salt

According to the general procedures A and B, starting from compound G1 and 2-tert-butylphenol. LC-MS: Rₜ=0.93 min, ES+=580.20.

### Example 3

### (rac.)-(1R*, 5S*)-3-Acetyl-7-{3-[2-(2,3,6-trifluorophenoxy)ethyl]phenyl-3,9-diazabicyclo[3.3.1]non-6-ene-6-carboxylic acid methylphenethylamide formate salt

According to the general procedures A and B, starting from compound G1 and 2,3,6-trifluorophenol. LC-MS: Rₜ=0.82 min, ES+=578.11.

### Example 4

### (rac.)-(1R*, 5S*)-3-Acetyl-7-(3-[2-(2,5-difluorophenoxy)ethyl]phenyl}-3,9-diazabicyclo[3.3.1]non-6-ene-6-carboxylic acid methylphenethylamide formate salt

According to the general procedures A and B, starting from compound G1 and 2,5-difluorophenol. LC-MS: Rₜ=0.81 min, ES+=560.11.

### Example 5

### (rac.)-(1R*, 5S*)-3-Acetyl-7-[3-(2-o-tolyloxyethyl)phenyl]-3,9-diazabicyclo-[3.3.1]non-6-ene-6-carboxylic acid methylphenethylamide formate salt

According to the general procedures A and B, starting from compound G1 and 2-methylphenol. LC-MS: Rₜ=0.84 min, ES+=538.15.

### Example 6

### (rac.)-(1R*, 5S*)-3-Acetyl-7-{3-[2-(2,3-dichlorophenoxy)ethyl]phenyl}-3,9-diazabicyclo[3.3.1]non-6-ene-6-carboxylic acid methylphenethylamide formate salt

According to the general procedures A and B, starting from compound G1 and 2,3-dichlorophenol. LC-MS: Rₜ=0.87 min, ES+=592.06.

### Example 7

### (rac.)-(1R*, 5S*)-3-Acetyl-7-{3-[2-(2-chloro-5-methylphenoxy)ethyl]phenyl}-3,9-diazabicyclo[3.3.1]non-6-ene-6-carboxylic acid methylphenethylamide formate salt

According to the general procedures A and B, starting from compound G1 and 2-chloro-5-methylphenol. LC-MS: Rₜ=0.87 min, ES+=572.11.

### Example 8

### (rac.)-(1R*, 5S*)-3-Acetyl-7-{3-[2-(3-chlorophenoxy)ethyl]phenyl}-3,9-diazabicyclo[3.3.1]non-6-ene-6-carboxylic acid methylphenethylamide formate salt

According to the general procedures A and B, starting from compound G1 and 3-chlorophenol. LC-MS: Rₜ=0.85 min, ES+=558.11.

### Example 9

### (rac.)-(1R*, 5S*)-(3-Acetyl-7-{3-[2-(2-bromo-5-fluorophenoxy)ethyl]phenyl}-3,9-diazabicyclo[3.3.1]non-6-ene-6-carboxylic acid methylphenethylamide formate salt

According to the general procedures A and B, starting from compound G1 and 2-bromo-5-fluorophenol.

### Example 10

### (rac.)-(1R*, 5S*)-3-Acetyl-7-{3-[3-(2-bromo-5-fluorophenoxy)propyl]phenyl}-3,9-diazabicyclo[3.3.1]non-6-ene-6-carboxylic acid methylphenethylamide formate salt

According to the general procedures A and B, starting from compound G2 and 2-bromo-5-fluorophenol. LC-MS: Rₜ=0.89 min, ES+=636.03.

### Example 11

### (rac.)-(1R*, 5S*)-3-Acetyl-7-{3-[2-(2-chlorophenoxy)propyl]phenyl}-3,9-diazabicyclo[3.3.1]non-6-ene-6-carboxylic acid methylphenethylamide formate salt

According to the general procedures A and B, starting from compound G2 and 2-chlorophenol. LC-MS: Rₜ=0.86 min, ES+=572.10.

### Example 12

### (rac.)-(1R*, 5S*)-3-Acetyl-7-{3-[2-(2-tert-butylphenoxy)propyl]phenyl}-3,9-diazabicyclo[3.3.1]non-6-ene-6-carboxylic acid methylphenethylamide formate salt

According to the general procedures A and B, starting from compound G2 and 2-tert-butylphenol. LC-MS: Rₜ=0.97 min, ES+=594.23.

### Example 13

### (rac.)-(1R*, 5S*)-3-Acetyl-7-{3-[2-(2,3,6-trifluorophenoxy)propyl]phenyl}-3,9-diazabicyclo[3.3.1]non-6-ene-6-carboxylic acid methylphenethylamide formate salt

According to the general procedures A and B, starting from compound G2 and 2,3,6-trifluorophenol. LC-MS: Rₜ=0.85 min, ES+=592.13.

### Example 14

### (rac.)-(1R*, 5S*)-3-Acetyl-7-{3-[2-(2,5-difluorophenoxy)propyl]phenyl)-3,9-diazabicyclo[3.3.1]non-6-ene-6-carboxylic acid methylphenethylamide formate salt

According to the general procedures A and B, starting from compound G2 and 2,5-difluorophenol. LC-MS: Rₜ=0.84 min, ES+=574.14.

### Example 15

### (rac.)-(1R*, 5S*)-3-Acetyl-7-[3-(2-O-tolyloxypropyl)phenyl]-3,9-diazabicyclo-[3.3.1]non-6-ene-6-carboxylic acid methylphenethylamide formate salt

According to the general procedures A and B, starting from compound G2 (50 mg) and 2-methylphenol. LC-MS: Rₜ=0.88 min, ES+=552.17.

### Example 16

### (rac.)-(1R*, 5S*)-3-Acetyl-7-(3-[2-(2,3-dichlorophenoxy)propyl]phenyl}-3,9-diazabicyclo[3.3.1]non-6-ene-6-carboxylic acid methylphenethylamide formate salt

According to the general procedures A and B, starting from compound G2 and 2,3-dichlorophenol. LC-MS: Rₜ=0.91 min, ES+=606.08.

### Example 17

### (rac.)-(1R*, 5S*)-3-Acetyl-7-{3-[2-(2-chloro-5-methylphenoxy)propyl]-phenyl}-3,9-diazabicyclo[3.3.1]non-6-ene-6-carboxylic acid methylphenethylamide formate salt

According to the general procedures A and B, starting from compound G2 and 2-chloro-5-methylphenol. LC-MS: Rₜ=0.90 min, ES+=586.12.

### Example 18

### (rac.)-(1R*, 5S*)-3-Acetyl-7-{3-[2-(3-chlorophenoxy)propyl]phenyl}-3,9-diazabicyclo[3.3.1]non-6-ene-6-carboxylic acid methylphenethylamide formate salt

According to the general procedures A and B, starting from compound X2 (50 mg) and 3-chlorophenol. LC-MS: Rₜ=0.88 min, ES+=572.12.

### Example 19

### (rac.)-(1R*, 5S*)-3-Acetyl-7-{3-[2-(4-chlorophenoxy)propyl]phenyl}-3,9-diazabicyclo[3.3.1]non-6-ene-6-carboxylic acid methylphenethylamide formate salt

According to the general procedures A and B, staring from compound G2 and 4-chlorophenol. LC-MS: Rₜ=0.88 min, ES+=572.11.

### Example 20

### (rac.)-(1R*, 5S*)-7-{3-[2-(2,6-Dichloro-4-methylphenoxy)ethoxy]phenyl}-3,9-diazabicyclo[3.3.1]non-6-ene-6-carboxylic acid cyclopropyl-(3-methoxy-2-methylbenzyl)amide

According to the general procedures A and B, starting from compound G3 and 2,6-dichloro-4-methylphenol. LC-MS: Rₜ=0.88 min, ES+=572.11.

### Example 21

### (rac.)-(1R*, 5S*)-7-{3-[4-(2-Fluoro-3-trifluoromethylphenoxy)butoxy]-phenyl}-3,9-diazabicyclo[3.3.1]non-6-ene-6-carboxylic acid cyclopropyl-(3-methoxy-2-methylbenzyl)amide

According to the general procedures A and B, starting from compound G4 and 2-fluoro-3-trifluoromethylphenol. LC-MS: Rₜ=0.88 min, ES+=572.11.

### Example 22

### (rac.)-(1R*, 5S*)-7-{3-[4-(2,6-Dichloro-4-methylphenoxy)butoxy]phenyl}-3,9-diazabicyclo[3.3.1]non-6-ene-6-carboxylic acid cyclopropyl-(3-methoxy-2-methylbenzyl)amide

According to the general procedures A and B, starting from compound G4 and 2,6-dichloro-4-methylmethylphenol. LC-MS: Rₜ=0.88 min, ES+=572.11.

### Example 23

### (rac.)-(1R*, 5S*)-7-{3-[4-(2-Chloro-6-fluoro-3-methylphenoxy)butoxy]-phenyl}-3,9-diazabicyclo[3.3.1]non-6-ene-6-carboxylic acid cyclopropyl-(3-methoxy-2-methylbenzyl)amide

According to the general procedures A and B, starting from compound G4 and 2-chloro-6-fluoro-3-methylphenol. LC-MS: Rₜ=0.88 min, ES+=572.11.

The following assay was carried out in order to determine the activity of the compounds of general formula I and their salts.

Inhibition of Human Recombinant Renin by the Compounds of the Invention

The enzymatic in vitro assay was performed in 384-well polypropylene plates (Nunc). The assay buffer consisted of 10 mM PBS (Gibco BRL) including 1 mM EDTA and 0.1% BSA. The incubates were composed of 50 μL per well of an enzyme mix and 2.5 μL of renin inhibitors in DMSO. The enzyme mix was premixed at 4° C. and consists of the following components:

human recombinant renin (0.16 ng/mL) • synthetic human angiotensin(1-14) (0.5 μM)

hydroxyquinoline sulfate (1 mM)

The mixtures were then incubated at 37° C. for 3 h.

To determine the enzymatic activity and its inhibition, the accumulated Ang I was detected by an enzyme immunoassay (EIA) in 384-well plates (Nunc). 5 μL of the incubates or standards were transferred to immuno plates which were previously coated with a covalent complex of Ang I and bovine serum albumin (Ang 1-BSA). 75 μL of Ang I-antibodies in essaybuffer above including 0.01% Tween 20 were added and a primary incubation made at 4° C. overnight. The plates were washed 3 times with PBS including 0.01% Tween 20, and then incubated for 2 h at rt with an antirabbit-peroxidase coupled antibody (WA 934, Amersham). After washing the plates 3 times, the peroxidase substrate ABTS (2.2′-azino-di-(3-ethyl-benzthiazolinsulfonate), was added and the plates incubated for 60 min at room temperature. After stopping the reaction with 0.1 M citric acid pH 4.3 the plate was evaluated in a microplate reader at 405 nm. The percentage of inhibition was calculated of each concentration point and the concentration of renin inhibition was determined that inhibited the enzyme activity by 50% (IC₅₀). The IC₅₀-values of all compounds tested are below 10 μM.

1. A compound of formula I 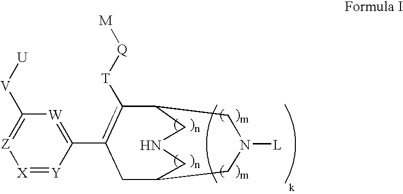
wherein
Z, Y, X and W represent independently a nitrogen atom or a —CH— group; at least two of the Z, Y, X and W represent a —CH— group;
V represents a bond; —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; or —CH₂—CH₂-A-CH₂—CH₂—B—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; or —COO—;
Q represents lower alkylene; lower alkenylene;
M represents hydrogen; cycloalkyl; aryl; heterocyclyl; heteroaryl;
L represents —R³; —COR³; —COOR³; —CONR²R³; —SO₂R³; —SO₂NR²R³; —COCH(Aryl)₂;
R¹ represents hydrogen; lower alkyl; lower alkenyl; lower alkinyl; cycloalkyl; aryl; cycloalkyl-lower alkyl;
R² and R²′ independently represent hydrogen; lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted or mono-, di- or trisubstituted with hydroxy, —OCOR², —COOR², lower alkoxy, cyano, —CONR²R²′, —CO-morpholin-4-yl, —CO-((4-loweralkyl)piperazin-1-yl), —NH(NH)NH₂, —NR⁴R⁴′ or lower alkyl, with the proviso that a carbon atom is attached at the most to one heteroatom in case this carbon atom is sp3-hybridized;
R⁴ and R⁴′ independently represent hydrogen; lower alkyl; cycloalkyl; cycloalkyl-lower alkyl; hydroxy-lower alkyl; —COOR²; —CONH₂;
k is the integer 0 or 1;
m and n represent the integer 0 or 1, with the proviso that in case m represents the integer 1, n is the integer 0; in case n represents the integer 1, m is the integer 0; in case k represents the integer 0, n represents the integer 0;
p is the integer 1, 2, 3 or 4;
r is the integer 1, 2, 3, 4, 5, or 6;
s is the integer 1, 2, 3, 4, or 5;
t is the integer 1, 2, 3, or 4;
u is the integer 1, 2, or 3; and
v is the integer 2, 3, or 4,
or optically pure enantiomers, racemates, diastereomers, mixtures of diastereomers, diastereomeric racemates, mixtures of diastereomeric racemates, or the meso-form of the compound; or pharmaceutically acceptable salts, solvent complexes or morphological forms of the compound.

2. The compound of formula I according to claim 1, wherein
k is 1
n is 0 and
m is 1,
or optically pure enantiomers, racemates, diastereomers, mixtures of diastereomers, diastereomeric racemates, mixtures of diastereomeric racemates, or the meso-form of the compound; or pharmaceutically acceptable salts, solvent complexes or morphological forms of the compound.

3. The compound of formula I according to claim 1, wherein
L represents H; —COR³″; —COOR³″; —CONR²″R³″; and
R²″ and R³″ represent independently lower alkyl; lower cycloalkyl-lower alkyl, which lower alkyl and lower cycloalkyl-lower alkyl are unsubstituted or mono-substituted with halogen, —CN, —OH, —OCOCH₃, —CONH₂, —COOH, or —NH₂, with the proviso that a carbon atom is attached at the most to one heteroatom in case this carbon atom is sp3-hybridized,
or optically pure enantiomers, racemates, diastereomers, mixtures of diastereomers, diastereomeric racemates, mixtures of diastereomeric racemates, or the meso-form of the compound; or pharmaceutically acceptable salts, solvent complexes or morphological forms of the compound.

4. The compound of formula I according to claim 1, wherein
T represents —CONR¹—;
Q represents methylene; and
M represents aryl or heteroaryl,
or optically pure enantiomers, racemates, diastereomers, mixtures of diastereomers, diastereomeric racemates, mixtures of diastereomeric racemates, or the meso-form of the compound; or pharmaceutically acceptable salts, solvent complexes or morphological forms of the compound.

5. The compound of formula I according to claim 1, wherein
Z, Y, X and W represent —CH—,
or optically pure enantiomers, racemates, diastereomers, mixtures of diastereomers, diastereomeric racemates, mixtures of diastereomeric racemates, and the meso-form of the compound; or pharmaceutically acceptable salts, solvent complexes or morphological forms of the compound.

6. The compound of formula I according to claim 1, wherein
U is a mono-, di-, or trisubstituted phenyl or heteroaryl, the substituents being selected from the group consisting of halogen, lower alkyl, lower alkoxy, and CF₃,
or optically pure enantiomers, mixtures of enantiomers such as racemates, diastereomers, mixtures of diastereomers, diastereomeric racemates, mixtures of diastereomeric racemates, or the meso-form of the compound; or pharmaceutically acceptable salts, solvent complexes or morphological forms of the compound.

7. The compound according to claim 1 selected from the group consisting of
(rac.)-(1R*,5S*)-(3-acetyl-7-{3-[2-(2-bromo-5-fluorophenoxy)ethyl]phenyl}-3,9-diazabicyclo[3.3.1]non-6-ene-6-carboxylic acid methylphenethylamide;
(rac.)-(1R*,5S*)-3-acetyl-7-{3-[2-(2-chlorophenoxy)ethyl]phenyl}-3,9-diazabicyclo[3.3.1]non-6-ene-6-carboxylic acid methylphenethylamide;
(rac.)-(1R*,5S*)-3-acetyl-7-{3-[2-(2-tert-butylphenoxy)ethyl]phenyl}-3,9-diazabicyclo[3.3.1]non-6-ene-6-carboxylic acid methylphenethylamide;
(rac.)-(1R*,5S*)-3-acetyl-7-{3-[2-(2,3,6-trifluorophenoxy)ethyl]phenyl}-3,9-diazabicyclo[3.3.1]non-6-ene-6-carboxylic acid methylphenethylamide;
(rac.)-(1R*,5S*)-3-acetyl-7-{3-[2-(2,5-difluorophenoxy)ethyl]phenyl}-3,9-diazabicyclo[3.3.1]non-6-ene-6-carboxylic acid methylphenethylamide;
(rac.)-(1R*,5S*)-3-acetyl-7-[3-(2-o-tolyloxyethyl)phenyl]-3,9-diazabicyclo-[3.3.1]non-6-ene-6-carboxylic acid methylphenethylamide;
(rac.)-(1R*,5S*)-3-acetyl-7-{3-[2-(2,3-dichlorophenoxy)ethyl]phenyl}-3,9-diazabicyclo[3.3.1]non-6-ene-6-carboxylic acid methylphenethylamide;
(rac.)-(1R*,5S*)-3-acetyl-7-{3-[2-(2-chloro-5-methylphenoxy)ethyl]phenyl}-3,9-diazabicyclo[3.3.1]non-6-ene-6-carboxylic acid methylphenethylamide;
(rac.)-(1R*,5S*)-3-acetyl-7-{3-[2-(3-chlorophenoxy)ethyl]phenyl}-3,9-diazabicyclo[3.3.1]non-6-ene-6-carboxylic acid methylphenethylamide;
(rac.)-(1R*,5S*)-3-acetyl-7-{3-[3-(2-bromo-5-fluorophenoxy)propyl]phenyl}-3,9-diazabicyclo[3.3.1]non-6-ene-6-carboxylic acid methylphenethylamide;
(rac.)-(1R*,5S*)-3-acetyl-7-{3-[2-(2-chlorophenoxy)propyl]phenyl}-3,9-diazabicyclo[3.3.1]non-6-ene-6-carboxylic acid methylphenethylamide;
(rac.)-(1R*,5S*)-3-acetyl-7-{3-[2-(2-tert-butylphenoxy)propyl]phenyl}-3,9-diazabicyclo[3.3.1]non-6-ene-6-carboxylic acid methylphenethylamide;
(rac.)-(1R*,5S*)-3-acetyl-7-{3-[2-(2,3,6-trifluorophenoxy)propyl]phenyl}-3,9-diazabicyclo[3.3.1]non-6-ene-6-carboxylic acid methylphenethylamide;
(rac.)-(1R*,5S*)-3-acetyl-7-{3-[2-(2,5-difluorophenoxy)propyl]phenyl}-3,9-diazabicyclo[3.3.1]non-6-ene-6-carboxylic acid methylphenethylamide;
(rac.)-(1R*,5S*)-3-acetyl-7-[3-(2-o-tolyloxypropyl)phenyl]-3,9-diazabicyclo-[3.3.1]non-6-ene-6-carboxylic acid methylphenethylamide;
(rac.)-(1R*,5S*)-3-acetyl-7-{3-[2-(2,3-dichlorophenoxy)propyl]phenyl}-3,9-diazabicyclo[3.3.1]non-6-ene-6-carboxylic acid methylphenethylamide;
(rac.)-(1R*,5S*)-3-acetyl-7-{3-[2-(2-chloro-5-methylphenoxy)propyl]phenyl}-3,9-diazabicyclo[3.3.1]non-6-ene-6-carboxylic acid methylphenethylamide;
(rac.)-(1R*,5S*)-3-acetyl-7-{3-[2-(3-chlorophenoxy)propyl]phenyl}-3,9-diazabicyclo[3.3.1]non-6-ene-6-carboxylic acid methylphenethylamide;
(rac.)-(1R*,5S*)-3-acetyl-7-{3-[2-(4-chlorophenoxy)propyl]phenyl}-3,9-diazabicyclo[3.3.1]non-6-ene-6-carboxylic acid methylphenethylamide;
(rac.)-(1R*,5S*)-7-{3-[2-(2,6-dichloro-4-methylphenoxy)ethoxy]phenyl}-3,9-diazabicyclo[3.3.1]non-6-ene-6-carboxylic acid cyclopropyl-(3-methoxy-2-methylbenzyl)amide;
(rac.)-(1R*,5S*)-7-{3-[4-(2-fluoro-3-trifluoromethylphenoxy)butoxy]phenyl}-3,9-diazabicyclo[3.3.1]non-6-ene-6-carboxylic acid cyclopropyl-(3-methoxy-2-methylbenzyl)amide;
(rac.)-(1R*,5S*)-7-{3-[4-(2,6-dichloro-4-methylphenoxy)butoxy]phenyl}-3,9-diazabicyclo[3.3.1]non-6-ene-6-carboxylic acid cyclopropyl-(3-methoxy-2-methylbenzyl)amide; and
(rac.)-(1R*,5S*)-7-{3-[4-(2-chloro-6-fluoro-3-methylphenoxy)butoxy]phenyl}-3,9-diazabicyclo[3.3.1]non-6-ene-6-carboxylic acid cyclopropyl-(3-methoxy-2-methylbenzyl)amide.

8. A pharmaceutical composition comprising at least one compound of claim 1 and a carrier and/or an adjuvant.

9. A method for the treatment or prophylaxis of RAS-associated diseases comprising hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, or complications of treatment with immunosuppressive agents after organ transplantation, which method comprises administering the compound according to claim 1 to a subject.

10. (canceled)

11. (canceled)

12. A pharmaceutical composition comprising at least one compound of claim 7 and a carrier and/or adjuvant.

13. A method for the treatment or prophylaxis of RAS-related diseases comprising hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, or complications of treatment with immunosuppressive agents after organ transplantation, which method comprises administering the compound according to claim 7 to a subject.
